(19) United States
(12) Patent Application Publication
(10) Pub. No.: US 2019/0282593 A1
(43) Pub. Date: Sep. 19, 2019

(54) COMPOSITIONS OF CIDOFOVIR AND METHODS OF USE

(71) Applicant: INITIUM INNOVATIONS, INC., Alvaton, KY (US)

(72) Inventor: James ALLRED, Alvaton, KY (US)

(21) Appl. No.: 16/354,609

(22) Filed: Mar. 15, 2019

Related U.S. Application Data
(60) Provisional application No. 62/643,382, filed on Mar. 15, 2018.

(51) Int. Cl. A61K 31/675; A61K 9/00; A61K 47/32; A61P 35/00; A61P 17/12; A61P 31/20; A61K 9/70; A61K 47/38

(52) U.S. Cl. CPC A61K 31/675; A61K 9/0021; A61K 47/32; A61K 47/38; A61P 17/12; A61P 31/20; A61K 9/7023; A61P 35/00

(57) ABSTRACT

Disclosed herein are compositions of cidofovir and methods of use. In some embodiments, the biodegradable sustained release compositions comprise an effective amount of cidofovir. Also disclosed herein are methods to treat a human papillomavirus (HPV)-associated disease in a subject by biodegradable sustained release compositions of cidofovir.

### CLAIM OF PRIORITY

This application claims priority to U.S. provisional Application No. 62/643,382, filed on Mar. 15, 2018, entitled “Compositions of Cidofovir and Methods of Use,” and is incorporated herein by reference.

### SUMMARY

Disclosed herein are compositions of cidofovir and methods of use. In some embodiments, the biodegradable sustained release composition comprises an effective amount of cidofovir. In some embodiments, the biodegradable sustained release composition may be an injection or an implant, such as an epidermal implant or a dermal implant, or an implant below the mucosal surface. In some embodiments, the biodegradable sustained release composition may be in the form of biodegradable microneedle array. The biodegradable microneedle array may comprise a base portion, and a plurality of microneedles extending from the base portion, and cidofovir is disposed within the plurality of microneedles.

In some embodiments, a method of treating a human papillomavirus (HPV)-associated disease in a subject comprises administering to the subject in need thereof a biodegradable sustained release composition comprising an effective amount of cidofovir.

In some embodiments, a method of preventing or ameliorating a human papillomavirus (HPV)-associated disease in a subject comprises administering to the subject in need thereof a biodegradable sustained release composition comprising an effective amount of cidofovir.

### DETAILED DESCRIPTION

As used in this application and in the claims, the singular forms “a,” “an,” and “the” include the plural forms unless the context clearly dictates otherwise. Additionally, the term “includes” means “comprises.”

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans In some embodiments, the patient or subject is an adult, child or infant In some embodiments, the patient or subject is a human.

The term “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

An “effective amount” of a composition is a predetermined amount calculated to achieve the desired effect, i.e., to ameliorate, prevent or improve an unwanted condition, disease or symptom of a patient. The activity contemplated by the present methods may include both therapeutic and/or prophylactic treatment, as appropriate. The specific dose of the agent administered according to this invention is to obtain therapeutic and/or prophylactic effects. These will, of course, be determined by the particular circumstances surrounding the case, including, for example, the compound administered, the route of administration, and the condition being treated. The effective amount administered may be determined by a physician in the light of the relevant circumstances, including the condition to be treated, the choice of the effective agent to be administered, and the chosen route of administration.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “treating” is used herein, for instance, in reference to methods of treating a skin disorder or a systemic condition, and generally includes the administration of a therapeutic compound or composition which reduces the frequency of, or delays the onset of, symptoms of a medical condition or enhance the texture, appearance, color, sensation, or hydration of the intended tissue treatment area of the tissue surface in a subject relative to a subject not receiving the therapeutic compound or composition. This can include reversing, reducing, or arresting the symptoms, clinical signs, and underlying pathology of a condition in a manner to improve or stabilize a subject's condition.

### Compositions

Disclosed herein are biodegradable sustained release compositions of cidofovir and methods of using the same. Sustained (or controlled) release refers to the gradual release of cidofovir from the composition over a period of time. While there may be an initial burst phase, in some embodiments, it is preferred that the release display relatively linear kinetics, thereby providing a constant supply of cidofovir over the release period. The release period may vary from several hours to several months, depending upon the cidofovir concentration in the composition and its intended use. It is desirable that the release of cidofovir from the composition over the treatment period be relatively constant. The duration of the release period may be controlled by, inter alia, the composition of the biocompatible polymer matrix, the concentration of cidofovir, the locus of administration, and addition of release profile modifying agents.

In some embodiments, the biodegradable sustained release compositions of cidofovir comprise a biocompatible polymeric matrix selected from the group consisting of carboxy methyl cellulose (CMC) polymer, polylactide (PLA), polyglycolide (PGA), polybutylene succinate (PBS), polyhydroxyalkanoate (PHA), polycaprolactone acid lactone (PCL), polyhydroxybutyrate (PHB), polyvinyl pyrrolidone (PVP), polyvinyl alcohol (PVA), cyclic-olefin copolymer (COC), PHB and PHV copolymer (PHBV), poly lactic acid (PLA)-polyethylene glycol (PEG) copolymers (PLEG), and combinations thereof Examples of other non-limiting polymeric matrix materials that may be used include silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof

Biodegradable sustained release compositions are typically formulated with cidofovir loading of about 2% to about 85% by weight of the total composition. For example, the sustained release composition may contain a polymer matrix and about 2% to about 20%, about 2% to about 30%, about 2% to about 40%, about 2% to about 50%, about 2% to about 60%, about 2% to about 70%, about 2% to about 80%, or about 2% to about 85% of cidofovir by weight of the total composition. The sustained release compositions may contain cidofovir, a prodrug of cidofovir, and combinations thereof.

In some embodiments, the ratio of the polymer matrix to cidofovir may be from about 0.001:1 weight % to about 9:1 weight %, about 0.001:1 weight % to about 5:1 weight %, or about 0.001:1 weight % to about 0.05:1 weight %. Specific examples include about 0.001:1 weight %, about 0.005:1 weight %, about 0.01:1 weight %, about 0.05:1 weight %, about 0.1:1 weight %, about 0.5:1 weight %, about 1:1 weight %, about 2:1 weight %, about 3:1 weight %, about 4:1 weight %, about 9:1 weight %, and ranges between any of the two values.

In some embodiments, the biodegradable sustained release composition may release cidofovir immediately and rapidly after implant or injection (immediate release), and continues thereafter for a period of time (sustained release). The immediate release period may be, for example, from 1 minute to 5 minutes, from 1 minute to 10 minutes, from 1 minute to 30 minutes, from 1 minute to 60 minutes, from 1 minute to 3 hours, from 1 minute to 6 hours, or from 1 minute to 24 hours, from 1 minute to 2 days, and from 1 minute to 3 days. In other embodiments, the sustained release period may be for about 2 days to about 4 days, about 2 days to about 7 days, about 1 week to about 2 weeks, about 1 week to about 3 weeks, about 1 week to about 1 month, about 1 week to about 2 months, about 1 week to about 3 months, about 1 week to about 4 months, about 1 week to about 5 months, about 1 week to about 6 months, about 1 week to about 9 months, about 1 week to about 12 months, or about 1 day to about 12 months. During this sustained release period, drug delivery proceeds at a near constant rate. For example, for a period of about 1 week to 2 weeks, about 50% of drug within the composition may be released. In other embodiments, for a period of about 1 week to 3 weeks, about 50% of drug will be released. In additional embodiments, for a period of about 1 week to 4 weeks, about 50% of drug will be released. In further embodiments, for a period of about 1 week to 6 weeks, about 50% of drug will be released. In further embodiments, for a period of about 1 week to 8 weeks, about 100% of drug will be released.

In other embodiments, the biodegradable sustained release composition may release cidofovir after a lag period after implantation or injection, such as a lag period of about 1 day, about 3 days, about 1 week, about 2 weeks, about 3 weeks, or about 4 weeks. Once cidofovir begins to release from the composition, the release process may continue for additional time period (sustained release period). The sustained release period may be for about 2 days to about 4 days, about 2 days to about 7 days, about 1 week to about 2 weeks, about 1 week to about 3 weeks, about 1 week to about 1 month, about 1 week to about 2 months, about 1 week to about 3 months, about 1 week to about 4 months, about 1 week to about 5 months, about 1 week to about 6 months, about 1 week to about 9 months, about 1 week to about 12 months, or about 1 day to about 12 months.

In some embodiments, the release of cidofovir may not be at a constant rate. For example, cidofovir may release may occur at a faster rate initially, for a period of 1-2 weeks, and later slow down. In other embodiments, cidofovir release may occur at a slow rate initially, and later occur at a faster rate.

In some embodiments, the biodegradable sustained release compositions may be implantable (e.g., an implantable device) or injectable. In some embodiments, the sustained release composition be in the form of depot injections, tablets, rod-shaped structures, or cylindrical structures, and may be produced using an extrusion process, wherein the polymeric matrix is blended with cidofovir, melted, and extruded into rod-shaped structures. Rods are cut into individual implantable devices of the desired length, packaged, and sterilized prior to use. Other methods for encapsulating therapeutic compounds in implantable polymeric, nonerodible sustained release matrices are well known to those of skill in the art. Multiple implantable devices may be used, or the size and shape of the devices may be modified, to achieve a desired overall dosage.

In some embodiments, the biodegradable sustained release compositions can be in forms which include, but are not limited to, softgels, tablets, capsules, cachets, pellets, pills, powders and granules that may be injected or implanted into dermal, epidermal, or mucosal tissue.

In some embodiments, the biodegradable sustained release compositions may be in the form of biodegradable microneedle array. The biodegradable microneedle array may comprise a base portion, and a plurality of microneedles extending from the base portion, and cidofovir is disposed within the plurality of microneedles. The microneedles may be made of any biocompatible polymeric matrix disclosed herein.

In some embodiments, the plurality of microneedles are pre-formed to have different shapes and sizes. For example, in some embodiments, shape of the microneedle may be a pyramidal shaped, or cylindrical, or pillar shaped, and tip portion that generally tapers to a point. In some embodiments, the microneedle may have generally polygonal shape (for example, rectangular), and a tip portion that generally tapers to a point. The tip portion may be pyramidal in shape, and the exposed faces of the pyramid can be either flat or arcuate. The average tip diameter of the needles is about 1 μm to about 10 μm, about 1 μm to about 8 μm, about 1 μm to about 6 μm, or about 1 μm to about 4μm. In some embodiment, the width or the thickness of the microneedle may range from about 10 μm to about 1000 μm, about 10 μm to about 500 μm, about 10 μm to about 300 μm, about 10 μm to about 200 μm, or about 10 μm to about 100 μm. In some embodiments, the length of the microneedle may be from about 0.01 mm to about 5 mm, about 0.01 mm to about 4 mm, about 0.01 mm to about 2 mm, about 0.01 mm to about 1 mm, or about 0.01 mm to about 0.5 mm.

In some embodiments, the needles in the microneedle array may be arranged in a pattern, such as linear, circular, concentric, or irregular. In some embodiments, the needles may be arranged in a circular pattern with gap in the center such that when applied to a wart, the needles penetrate the skin around the wart.

In some embodiments, the microneedle array may be in the form of a patch. The microneedles are biosoluble and biodegradable. Thus the biodegradable microneedle dissolves in the skin after the patch is applied. In some embodiments, microneedles detach from patch when the patch is applied to a surface and removed, and then the microneedles dissolve. Having dissolved, the material will then be metabolized to give harmless end-products. The timescale for dissolving after applying the patch can vary, but dissolving will typically commence immediately after applying the patch (e.g. within 10 seconds) and may continue for e.g. up to 1 minute, 5 minutes, 10 minutes, 20 minutes, 30 minutes, 1 hour, 5 hours, 10 hours, or 24 hours, until the microneedle has fully dissolved. Materials with suitable in vivo dissolving kinetics are readily available and these can be varied and tested to determine appropriate concentrations etc. for any desired dissolution profile.

In some embodiments, the release period of cidofovir from the microneedles may be for example, from 1 minute to 5 minutes, from 1 minute to 10 minutes, from 1 minute to 30 minutes, from 1 minute to 60 minutes, from 1 minute to 3 hours, from 1 minute to 6 hours, or from 1 minute to 24 hours, when administered by a microneedle array. In other embodiments, the release period may be for about 2 days to about 4 days, about 2 days to about 7 days, about 1 week to about 2 weeks, about 1 week to about 3 weeks, or about 1 week to about 1 month.

In some embodiments, the patch may have a skin-facing inner face and an environment-facing outer face. The inner face may include an adhesive to facilitate adherence to a subject's skin. When present, it is preferably not present on the microneedles themselves i.e. the microneedles are adhesive-free. For example, a patch may have an additional backing which provides an outer adhesive margin for adhering the patch to skin e.g. as seen in sticking plasters or nicotine patches.

In some embodiments, the biodegradable sustained release compositions may further comprise pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The biodegradable sustained release pharmaceutical compositions disclosed herein may further contain a hydrogel. Non- limiting examples of hydrogels include methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), and hydroxyethylcarboxymethyl cellulose (HECMC).

### Methods

Also disclosed herein are methods of treating human papillomavirus (HPV) associated diseases. In some embodiments, a method of treating a human papillomavirus (HPV) associated disease in a subject comprises administering to the subject in need thereof a biodegradable sustained release composition comprising an effective amount of cidofovir.

In some embodiments, a method treating a human papillomavirus (HPV)-associated disease in a subject comprises administering an effective amount of cidofovir by a biodegradable microneedle array, wherein the biodegradable microneedle array comprises a base portion, and a plurality of microneedles extending from the base portion, and wherein the cidofovir is disposed within the plurality of microneedles.

In some embodiments, a method of preventing or ameliorating a human papillomavirus (HPV) associated disease in a subject comprises administering to the subject in need thereof a biodegradable sustained release composition comprising an effective amount of cidofovir.

In some embodiments, HPV-associated disease may be HPV-associated cancer, such as cervical intraepithelial neoplasia (CIN), cervical dysplasia, vaginal cancer, vaginal dysplasia, vulvar cancer, penile cancer, anal cancer, oral cancer, oropharyngeal cancer, oral epithelial hyperplasia, oral leucoplasias, vulval cancer, penile cancer, head and neck cancer, esophageal cancer, squamous cell carcinoma of the mucosa including the mouth, lips, tongue, buccal mucosa, palate, gingiva, nasopharynx, conjunctiva, vagina, cervix, and urethral mucosa, cutaneous squamous cell carcinoma, respiratory squamous cell carcinoma, verrucous carcinoma, atypical squamous cells of undetermined significance (ASCUS), laryngeal papilloma, oral papilloma, conjunctival papilloma, respiratory papillomatosis, maxiallary sinus papilloma, oral focal hyperplasia, and combinations thereof In some embodiments, HPV-associated disease may be conditions, such as genital warts, plantar warts, butcher's warts, flat warts, condyloma accuminata, epidermodysplasia verruciformis, mucosal warts, myrcemies, actinic keratoses, seborrheic keratoses, Verruca warts, plane warts, intermediary warts, Bowen's disease, and combinations thereof.

The methods and compositions described herein can be used to treat or prevent HPV-associated diseases associated with HPV types 16 and 18, as well as other HPV types including but not limited to HPV types 6, 11, 31, 33, 35, 39, 44, 45, 52, 53, 54, 56, 58, 61, 66, 61, 69, CP8304, CP141, MM4, MM7, and/or MM9. Conditions associated with specific HPV types include, for example, exophytic condyloma (HPV types 6 and 11), flat condyloma, especially of the cervix (HPV types 6, 11, 16, 18, and 31), giant condyloma (HPV types 6 and 11), cervical cancer (HPV types 16, 18, 31, 45, and 33), respiratory and conjunctival papillomatosis (HPV types 6 and 11), and high grade cervical intraepithelial neoplasia (HPV 31, 33, 35, 39, 44, 45, 52, 53, 54, 56, 58, 61, 66, 67, 69, CP8304, CP141, MM4, MM7, and MM9).

In some embodiments, the compositions and methods disclosed herein can also be used to treat cytomegalovirus (CMV)-associated diseases, such as nephritis, hepatitis, myocarditis, retinitis, encephalitis, retinitis, mononucleosis, pneumonitis, and combinations thereof.

In some embodiments, the compositions and methods disclosed herein can also be used to treat herpes simplex virus (HSV)-associated diseases, such as herpetic gingivostomatitis, herpes labialis, herpes genitalis, herpes whitlow, herpes gladiatorum, herpes encephalitis, herpes meningitis, herpes esophagitis, herpes keratitis, Bell's palsy, Mollaret's meningitis, herpesrugbeiorum, eczema herpeticum, herpetic neuralgia, post-herpetic neuralgia, and combinations thereof.

In some embodiments, the compositions and methods disclosed herein can also be used to treat other DNA viruses including varicella-zoster virus (VZV), Epstein-Barr virus (EBV), human herpesvirus type 6 (HHV-6) and equine and bovine herpesviruses, papovaviruses (human polyoma virus), adenoviruses, iridoviruses, hepadnaviruses, Molluscum contagiosum, and poxviruses.

In some embodiments, administration of the biodegradable sustained release compositions may be subcutaneous, intradermal, intraepidermal, delivery to or implantation/injection below the mucosal surface (such as, mouth, lips, tongue, buccal mucosa, palate, gingiva, nasopharynx, conjunctiva, vagina, cervix, and urethral mucosa), implantation/injection below the submucosal layer, delivery to airway epithelium, or any combination thereof

In some embodiments, a method treating a human papillomavirus (HPV)-associated genital warts, plantar warts, butcher's warts, flat warts, condyloma accuminata, epidermodysplasia verruciformis, mucosal warts, myrcemies, actinic keratoses, seborrheic keratosis, Verruca warts, plane warts, and intermediary warts, in a subject comprises administering a biodegradable sustained release composition comprising an effective amount of cidofovir, wherein the administration is performed intradermally or intra-epidermally

The dimensions of the sustained release compositions commensurate with the size and shape of the region selected as the site of administration and will not migrate from the insertion site following implantation, injection or other means of depot administration. The sustained release compositions may be rigid, or somewhat flexible so as to facilitate both insertion of the implant at the target site and accommodation of the implant. The sustained release compositions may be particles, sheets, patches, plaques, fibers, microcapsules and the like and may be of any size or shape compatible with the selected site of insertion.

In one embodiment, the sustained release composition may be administered by a catheter. In another embodiment, the sustained release composition may be administered by a syringe. The sustained release composition is formulated so that the composition can be readily implanted (e.g., by injection) into the desired location to form a mass that can remain in place for the period suitable for controlled release of cidofovir and for any additional benefit of mechanical support if applicable. The mechanical and rheological properties suitable for injectable depot compositions are known in the art. Typically, the polymer of the depot vehicle with particulates are present in an appropriate amount of solvent such that the depot composition can be so implanted.

An alternative embodiment of the invention provides for a rod depot implant. Other embodiments include a drug depot implant comprising a hollow depot, the hollow depot comprising cidofovir that provides a concentration gradient for targeted delivery at epidermis, at dermis, or between epidermis and dermis layer in a subject.

Depth of the deposition of cidofovir would vary based on body site and depending on the condition, such as wart size, and could range from 0.1 millimeters to 2 millimeters or more. Additionally, injection of the implant could be done vertically (i.e. injected perpendicular to the skin) or could be done from the side of the wart at a near parallel trajectory to the skin. In some embodiments the implant will be placed in the epithelium, or may span across the epithelium into underlying tissue such as the dermis, or in other embodiments it may pass through the epithelium and be implanted solely in the deeper tissues such as the dermis.

In some embodiments, administration of the biodegradable sustained release composition may be by a biodegradable microneedle array. Administration may be topical, transdermal, percutaneous, intradermal, intraepidermal, or to any mucosal surface, such as mucosa of the mouth, lips, tongue, buccal mucosa, palate, gingiva, nasopharynx, respiratory epithelium, conjunctiva, vagina, cervix, and urethral mucosa.

Microneedle arrays in the form of patches may be applied to the skin by simple manual application (e.g. as with a sticking plaster or with known skin patches) or may be applied using a spring-driven injector.

Microneedle arrays can be applied to a human tissue by a variety of methods including self or assisted application by human pressure (e.g., pushing with a forger or thumb), or with spring-loaded devices. To facilitate the ease and reproducibility of delivery of microneedle array devices, including tip-loaded microneedle arrays, an applicator device may be used. The applicator device is configured to convert high frequency electromagnetic oscillation into unidirectional mechanical resonance of the active head. This in turn enables multiple reproducible low amplitude and high frequency pressure strokes that facilitate insertion of the microneedles of microneedle arrays into the human tissue.

In some embodiments, the microneedle array may be a hollow needle, and sustained release compositions are administered through the central hollow bore. In some embodiments the needle may be attached to an endoscope, colonoscope, anoscope, or bronchiosope. In such embodiments, the microneedle array is part of a drug delivery device that includes a drug storage element. The drug storage element is a means for containing a sustained release formulation for release to and through the microneedle array, for transdermal administration of the formulation via the microneedle array. Preferably, the drug delivery device is in the form of a transdermal drug delivery patch. The drug delivery device typically includes means for causing the drug formulation to be released from the drug storage element, permitting the drug formulation to flow into or otherwise be transported through the channel of the microneedle. The release typically is to and through the apertures in the planar substrate and thus to the base end of the channel in the microneedle. A wide variety of release mechanisms for releasing the drug formulation from the drug storage element can be envisioned by those skilled in the art. These release mechanisms may utilize a mechanical force, heat, a chemical reaction, an electric field, a magnetic field, a pressure field, ultrasonic energy, vacuum, pressure, or a combination thereof.

In some embodiments, the effective dose of cidofovir that is released from the sustained release compositions may range from about 0.1 to 3000, 0.2 to 900, 0.3 to 800, 0.4 to 700, 0.5 to 600, 0.6 to 500, 70 to 400, 80 to 300, 90 to 200, or 100 to 150 micrograms/day. In other embodiments, the dose may range from approximately 10 to 20, 21 to 40, 41 to 80, 81 to 100, 101 to 130, 131 to 150, 151 to 200, 201 to 280, 281 to 350, 351 to 500, 501 to 1000 milligrams/day. In specific embodiments, the dose may be at least approximately 20 to 100, 40 to 100, 80 to 200, 100 to 500, 130 to 500, 200 to 1000, 280 to 1000, 400 to 1000, 500 to 2000, 750 to 2000, 1000 to 2000, or 2000 to 3000 micrograms/dose. In specific embodiments, the dose may be at least approximately 20 to 100, 25 to 100, 40 to 500, 50 to 500, 75 to 750, 100 to 1000, or 200 to 3000 milligrams/dose.

In some embodiments, the sustained release composition can be administered one, two, three, four, five or more times each day, and applying can be carried out for a period of at least 1 month, 2 months, 3 months, 4 months, 6 months, 8 months or 12 months. Frequency of application would vary from a one-time application (for extended release periods) to a daily applications (for short release periods).

In some embodiments, the biodegradable microneedle array patches may be administered once, as needed, once daily, twice daily, three times a day, once a week, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the method includes administering the sustained release composition as an adjuvant therapy. In some embodiments, the method includes administering the sustained release composition as a neo-adjuvant therapy. In some embodiments, the sustained release composition can be administered with other treatments, such as radiation therapy, chemotherapy, targeted therapy, gene therapy, cryotherapy, electrosurgery, laser treatment, vaccines, immunotherapy, or hormone therapy.

In some embodiments, the sustained release compositions may be co-administered with anti-cancer agents (such as, gemcitabine, taxanes, 5-FU, methotrexate, bleomycin, cisplatin, vincristine, vinblastine, altretamine, procarbazine, dacarbazine, temozolomide, etoposide, camptothecan analogs, doxorubicin, daunorubicin, epirubicin, mitoxantrone, idarubicin,), interferons, anti-viral agents (such as, acyclovir, imiquimod, trifluridine, idoxuridine, penciclovir, famciclovir, gancyclovir, valacyclovir, acyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir), anti-bacterial agents, and combination thereof

In some embodiments, the methods may include a variety of additional steps including, for example, cleaning the surface tissue at the site of applying and the like. In embodiments, the methods may further include ablation of the tissue surface before, during or after administration of the compositions described herein. In embodiments, tissue surface ablation may include electromagnetic radiation, laser, dermal abrasion, chemical peel, ultrasound, heating, cooling, or by a needle.

In some embodiments, prior to the administration of sustained release composition, the targeted lesion may or may not be prepared or altered prior to injection. Preparation of the lesion may include anesthesia through numbing creams or injections. They may also including paring the lesion where the most superficial aspect of the wart (such as a hypertrophic stratum corneum) of the lesion is removed via a blade or other method to allow penetration of cidofovir to metabolically active cells. In some embodiments, the surface may be pretreated with keratolytic agents. The term “keratolytic agent” is used herein to mean a compound which loosens and removes the stratum corneum of the skin, or alters the structure of the keratin layers of skin. Suitable keratolytic agents include but are not limited to N-acetylcysteine, azelaic acid, cresols, dihydroxy benzene compounds, such as resorcinol and hydroquinone, alpha-hydroxy acids, such as lactic acid and glycolic acid, tricyclic acid, phenol, pyruvic acid, resorcinol, sulfur, salicylic acid, retinoic acid, isoretinoic acid, retinol, retinal, urea and derivatives, esters, salts and mixtures thereof.

What is claimed is:

1. A method of treating a human papillomavirus (HPV)-associated disease in a subject comprising administering to the subject in need thereof a biodegradable sustained release composition comprising an effective amount of cidofovir.

2. The method of claim 1, wherein the HPV-associated disease is selected from the group consisting of cervical intraepithelial neoplasia (CIN), cervical dysplasia, vaginal cancer, vaginal dysplasia, vulvar cancer, penile cancer, anal cancer, oral cancer, head and neck cancer, oropharyngeal cancer, oral epithelial hyperplasia, oral leucoplasias, vulval cancer, penile cancer, squamous cell carcinoma, cutaneous squamous cell carcinoma, respiratory squamous cell carcinoma, verrucous carcinoma, esophageal cancer, laryngeal papilloma, oral papilloma, conjunctival papilloma, respiratory papillomatosis, maxiallary sinus papilloma, oral focal hyperplasia, atypical squamous cells of undetermined significance (ASCUS), genital warts, plantar warts, butcher's warts, flat warts, condyloma accuminata, epidermodysplasia verruciformis, mucosal warts, myrcemies, actinic keratoses, seborrheic keratoses, Verruca warts, plane warts, intermediary warts, Bowen's disease, and combinations thereof.

3. The method of claim 1, wherein the biodegradable sustained release composition further comprises a polymeric matrix selected from the group consisting of carboxy methyl cellulose (CMC) polymer, polylactide (PLA), polyglycolide (PGA), polybutylene succinate (PBS), polyhydroxyalkanoate (PHA), polycaprolactone acid lactone (PCL), polyhydroxybutyrate (PHB), polyvinyl pyrrolidone (PVP), polyvinyl alcohol (PVA), cyclic-olefin copolymer (COC), PHB and PHV copolymer (PHBV), poly lactic acid (PLA)-polyethylene glycol (PEG) copolymers (PLEG), and combinations thereof.

4. The method of claim 1, wherein the administration is by injection or implant.

5. The method of claim 1, wherein the sustained release period of the composition is from about 1 day to about 12 months.

6. The method of claim 1, wherein the biodegradable sustained release composition releases about 0.1 microgram to about 3000 micrograms of cidofovir per day.

7. The method of claim 1, wherein the cidofovir comprises about 2% to about 85% of the total weight of the biodegradable sustained release composition.

8. A method treating a human papillomavirus (HPV)-associated disease in a subject comprising:
administering an effective amount of cidofovir by a biodegradable microneedle array, wherein the biodegradable microneedle array comprises a base portion, and a plurality of microneedles extending from the base portion, and wherein the cidofovir is disposed within the plurality of microneedles.

9. The method of claim 8, wherein the HPV-associated disease is selected from the group consisting of cervical intraepithelial neoplasia (CIN), cervical dysplasia, vaginal cancer, vaginal dysplasia, vulvar cancer, penile cancer, anal cancer, oral cancer, head and neck cancer, oropharyngeal cancer, oral epithelial hyperplasia, oral leucoplasias, vulval cancer, penile cancer, squamous cell carcinoma, cutaneous squamous cell carcinoma, respiratory squamous cell carcinoma, verrucous carcinoma, esophageal cancer, laryngeal papilloma, oral papilloma, conjunctival papilloma, respiratory papillomatosis, maxiallary sinus papilloma, oral focal hyperplasia, atypical squamous cells of undetermined significance (ASCUS), genital warts, plantar warts, butcher's warts, flat warts, condyloma accuminata, epidermodysplasia verruciformis, mucosal warts, myrcemies, actinic keratoses, seborrheic keratoses, Verruca warts, plane warts, intermediary warts, Bowen's disease, and combinations thereof.

10. The method of claim 8, wherein the biodegradable microneedle array comprises a polymeric matrix selected from the group consisting of carboxy methyl cellulose (CMC) polymer, polylactide (PLA), polyglycolide (PGA), polybutylene succinate (PBS), polyhydroxyalkanoate (PHA), polycaprolactone acid lactone (PCL), polyhydroxybutyrate (PHB), polyvinyl pyrrolidone (PVP), polyvinyl alcohol (PVA), cyclic-olefin copolymer (COC), PHB and PHV copolymer (PHBV), poly lactic acid (PLA)-polyethylene glycol (PEG) copolymers (PLEG), and combinations thereof.

11. The method of claim 8, wherein the amount of cidofovir that is administered is about 20 micrograms to about 3000 micrograms per dose.

12. The method of claim 8, wherein the biodegradable microneedle array is a transdermal patch.

13. A biodegradable sustained release composition comprising a polymeric matrix and cidofovir, wherein the polymeric matrix is selected from the group consisting of carboxy methyl cellulose (CMC) polymer, polylactide (PLA), polyglycolide (PGA), polybutylene succinate (PBS), polyhydroxyalkanoate (PHA), polycaprolactone acid lactone (PCL), polyhydroxybutyrate (PHB), polyvinyl pyrrolidone (PVP), polyvinyl alcohol (PVA), cyclic-olefin copolymer (COC), and combinations thereof.

14. The biodegradable sustained release composition of claim 13, wherein the cidofovir comprises about 2% to about 85% of the total weight of the biodegradable sustained release composition.

15. The biodegradable sustained release composition of claim 13, wherein the ratio of the polymer matrix to cidofovir is from about 0.001:1 weight % to about 9:1 weight %.

16. The biodegradable sustained release composition of claim 13, wherein the biodegradable sustained release composition is configured to release about 0.1 microgram to about 3000 micrograms of cidofovir per day.

17. A biodegradable microneedle array comprising:
a base portion and a plurality of biodegradable microneedles extending from the base portion, and wherein cidofovir is disposed within the plurality of biodegradable microneedles.

18. The biodegradable microneedle array of claim 17, wherein the biodegradable microneedle array comprises a polymeric matrix selected from the group consisting of carboxy methyl cellulose (CMC) polymer, polylactide (PLA), polyglycolide (PGA), polybutylene succinate (PBS), polyhydroxyalkanoate (PHA), polycaprolactone acid lactone (PCL), polyhydroxybutyrate (PHB), polyvinyl pyrrolidone (PVP), polyvinyl alcohol (PVA), cyclic-olefin copolymer (COC), PHB and PHV copolymer (PHBV), poly lactic acid (PLA)-polyethylene glycol (PEG) copolymers (PLEG), and combinations thereof.

19. The biodegradable microneedle array of claim 17, wherein the average tip diameter of the needle is about 1 μm to about 10 μm.
